(19) United States
(12) Patent Application Publication
(10) Pub. No.: US 2012/0184808 A1
(43) Pub. Date: Jul. 19, 2012

(54) METHOD AND APPARATUS FOR DELIVERY INTO THE FETAL TRACHEA

(75) Inventor: Edmund Y. Yang, St. Louis, MO (US)

(73) Assignee: SAINT LOUIS UNIVERSITY, St. Louis, MO (US)

(21) Appl. No.: 13/352,466

(22) Filed: Jan. 18, 2012

Related U.S. Application Data
(60) Provisional application No. 61/434,195, filed on Jan. 19, 2011.

(51) Int. Cl. A61B 1/018

(52) U.S. Cl. 600/104

(57) ABSTRACT

An method and apparatus for using polymerizable hydrogels to cause tracheal obstruction (TO) for treatment of pulmonary hypoplasia disorders to improve lung development in-utero. Use of the compound can cause effective TO for the purposes of inducing lung growth.

### CROSS REFERENCE

This application claims the benefit of and priority to U.S. Provisional Patent Application Ser. No. 61/434,195 filed on Jan. 19, 2011 and entitled Method and Apparatus For Delivery Into The Fetal Trachea, which is incorporated by reference in its entirety herein.

### BACKGROUND

1. Field of Invention

This invention relates generally to pulmonary disorders in infants and, more particularly, to treatment of pulmonary hypoplasia.

2. Background Art

Before birth, a developing infant depends on the fetal circulation to supply oxygen and nutrients, and then eliminate CO₂ and other wastes via the placenta. The birth process causes dramatic changes in blood flow, bypassing the placenta, altering flow through the heart and major vessels, and opening the blood vessels within the lungs. With its first breath, an infant sets changes into motion which convert the circulatory system from that of an aquatic being to that of an air breather. As the lungs fill with air, resistance to blood flow through the lungs drops and normal blood flow begins. Two other important changes are the closure of the ductus arteriosus and of the foramen ovale. These two change the blood flow through the pulmonary artery and the heart respectively. If the changes above do not take place or only partially occur, it can create a condition known as persistent pulmonary hypertension or persistent fetal circulation.

Pulmonary Hypoplasia (P-Hyp) is the failure for lungs to develop in-utero. This often afflicts only one lung. Basically, the lungs do not have sufficient tissue and blood flow to adequately perform the oxygen/carbon dioxide exchange that the body requires to function. Approximately 15% of all peri-natal deaths are the result of pulmonary hypoplasia, making it the most common cause of death for neo-natal infants. There is no particular, singular cause for P-Hyp, but family histories and ultrasound findings may provide a warning that development is not progressing as it should.

Pulmonary hypoplasia or aplasia is part of the spectrum of malformations characterized by incomplete development of lung tissue. For lung development to proceed normally, physical space in the fetal thorax must be adequate, and amniotic fluid must be brought into the lung by fetal breathing movements, leading to distension of the developing lung. Several factors affect the volume and composition of the amniotic fluid. The pressure in the fetal trachea is normally about 2 mm Hg higher than in the amniotic fluid, thus preventing outflow of fetal lung fluid. Any alteration in the critical volume and pressure relationships of amniotic fluid in the trachea and lung during the canalicular stage of fetal lung development at 15-28 weeks' gestation can induce hypoplasia.

Pulmonary hypoplasia results in bronchi and alveoli that are intact. The pulmonary artery is usually small or, sometimes, absent. In pulmonary agenesis, the lung is absent, as are the bronchi, airways, and pulmonary vasculature. The right and left sides are affected equally, but the prognosis is worse if the right side is involved because of associated severe congenital malformations. The affected side has reduced volume, and patients have homogeneous opacification of the entire lung, with a mediastinal shift to the same side. Compensatory overinflation of the opposite lung and herniation and congenital malformation are associated findings. In pulmonary aplasia, the lung is absent, but a rudimentary blind ending bronchus is present.

Surrounding amniotic fluid is important for optimal fetal lung growth. Although the association between oligohydramnios and pulmonary hypoplasia is well documented, the underlying mechanisms for this phenomenon have not been fully elucidated. Several explanations have been put forward: 1) decreased space for lung growth as a result of pressure of the uterine wall on the fetal chest and abdomen; 2) restriction of fetal breathing movements by prolonged thoracic compression; and 3) increased efflux of lung liquid from the intrapulmonary space to the amniotic space, resulting in a decrease of intrapulmonary pressure. Oligohydramnios may result from a different cause, such as lack of fetal urinary production associated with renal abnormalities, severe fetal growth restriction, and ruptured membranes.

Causes of pulmonary hypoplasia include a wide variety of congenital malformations and other conditions in which pulmonary hypoplasia is a complication. These include congenital diaphragmatic hernia, congenital cystic adenomatoid malformation, fetal hydrnephrosis, caudal regression syndrome, mediastinal tumor, and sacrococcygeal teratoma with a large component inside the fetus. Large masses of the neck (such as cervical teratoma) also can cause pulmonary hypoplasia, presumably by interfering with the fetus's ability to fill its lungs. In the presence of pulmonary hypoplasia, the EXIT procedure to rescue a baby with a neck mass is not likely to succeed.

To avoid mortality from severe lung hypoplasia in association with diaphragmatic hernia or, fetal surgical intervention has been attempted. Most studies report a mortality rate of 25-30% in neonates with Cystic Adenomatoid Malformation (CAM). However, in other cystic lung lesions, most are clinically asymptomatic and may not need aggressive management. Risk factors for a poor outcome include the presence of hydrop fetalis, with a mortality rate as high as 80-90%. Other indicators include the type of CAM and its size. All of these factors reflect the degree of pulmonary compromise with lesions that result in varying degrees of pulmonary hypoplasia. Pulmonary hypoplasia may be primary, but it is usually secondary, manifested by small fetal thoracic volume caused by compression in the hemithorax due to structures such as abdominal contents in Congenital Diaphragmatic Hernia (CDH) or congenital anomalies such as Congenital Adenomatoid Malformation (CAM) or cysts.

Pressure appears to affect fetal lung growth. Specifically, airway distension may affect various developmental and signaling pathways such as receptor tyrosine kinase growth factors, homeobox genes, transcription factors, retinoid signaling, and oxidation reduction. Experimentally, tracheal occlusion in fetal animals induces lung growth. These encouraging observations in various animal models have led to application to human fetuses with CDH. The detrimental effect of compression of the lung by other tissue such as herniation of abdominal viscera in the thorax in CHD is further suggested by a case report of bilateral CDH with gastroschisis.

In fetuses with pulmonary hypoplasia, before delivery and depending on the underlying lesion, a few interventions can be performed to increase the fetal lung volume and improve lung development. Preterm rupture of membranes without signs of fetal distress or intrauterine infection is treated conservatively with or without tocolytics, antibiotics, and steroids in various combinations. Attempts have been made to seal the defect in the membranes by transcervically using “fibrin glue.” However, this technique requires a preliminary cerclage, increases the risk of infection, and has limited efficacy. Serial amnioinfusions are increasingly used in cases of preterm rupture of membranes at less than 32 weeks' gestation. This procedure, if successful, has been shown to decrease the risk of pulmonary hypoplasia and significantly improve perinatal outcome.

As survival for other pulmonary conditions has improved in recent years, pulmonary hypoplasia (P-Hyp, inadequately sized lungs) has become an increasingly important cause of neonatal morbidity and mortality. P-Hyp has been described as the most common anomaly in infants who die in the neonatal period. P-Hyp, as discussed above, has several causes, and it can often be suspected on the basis of historical factors or ultrasound findings during the current pregnancy. Appropriate resuscitation of infants with P-Hyp requires special considerations and techniques.

In patients with severe CAM who have an extremely poor prognosis, fetal surgery is possible in certain centers. A multidisciplinary team with expertise in fetal surgery should evaluate both the fetus and the pregnant mother. A major indication for fetal surgery is the presence of hydrops and a gestation of less than 32 weeks. Thoracocentesis can allow for drainage of fluid from the CAM, but the fluid usually rapidly reaccumulates. Intrauterine vesicoamniotic shunts and endoscopic ablation of posterior urethral valves are other techniques that are currently used in fetuses with urinary tract obstruction and pulmonary hypoplasia. With careful case selection, pulmonary hypoplasia is prevented, and postnatal renal and respiratory function is improved.

Given the prevalence of this disorder and the expected infant survival rate for the more severe cases and improved methodology is needed to treat this disorder.

### BRIEF SUMMARY OF INVENTION

One embodiment of the invention is a method and apparatus for using polymerizable hydrogels to cause tracheal obstruction (TO) for treatment of pulmonary hypoplasia disorders to improve lung development in-utero. Use of the compound can cause effective TO for the purposes of inducing lung growth. Percutaneous fetal endoluminal tracheal occlusion (FETO) with a balloon, inserted at 26-28 weeks' gestation, has been considered for infants with isolated CDH with poor prognosis, but with little success. The stability of the balloon was problematic and subsequent removal proved problematic.

The method and apparatus of one embodiment of the invention proposes to use hydrogel as a new clinical method for TO in fetuses with lungs too small to survive (pulmonary hypoplasia). A commercially available hydrogel can be utilized, for example a hydrogel called Duraseal (owned by Coviden), however, a specialized hydrogel could be developed. Delivery of the compound into the fetal trachea must be effective, because when injecting the Duraseal into a fetus with severe congenital diaphragmatic hernia (CDH) and pulmonary hypoplasia at 26 weeks gestation, the Duraseal may not flow into the lungs and most of it may come out of the fetal lungs into the mouth and therefore, the TO, which can be monitored by ultrasound, may have too short of a life span.

One embodiment of the invention proposes placing or passing a balloon catheter through and into the fetal lung and passing an injection catheter through. For example a commercially available, long 9Fr vascular introducer can be utilized that can be advanced over the fetoscope into the fetal trachea. This can serve as a stiff access port to the fetal lung. The fetoscope can be steered into the fetal trachea, then the introducer can slide over the scope into the trachea. The scope can be removed and an 8.5Fr cholangiogram catheter can be placed into the trachea. The balloon can be inflated within the trachea to serve as a stopper to prevent the Duraseal from leaking out of the airway. The Duraseal can be injected below the balloon and allowed to polymerize.

Using this method and apparatus with an infant, the testing followed the infant for several weeks and saw excellent lung growth for approximately 3-5 weeks after the operation. When the infant was delivered at 34 weeks, there was no evidence of Duraseal in the airways.

One embodiment of the invention can comprise several components including, (1) the use a working sheath as a mechanical means of accessing the fetal trachea for drug or device deployment; (2) the use of a temporary occlusion balloon catheter to allow deeper penetration of drug or device delivery into the fetal trachea and lung; (3) the use of polymerizable drugs or compounds injected into the fetal lung below an occlusive balloon so as to achieve a clinical goal, whether that be lung growth, drug delivery or other purposes.

Percutaneous fetal endoluminal tracheal occlusion (FETO) with a balloon and injection of a hydrogel, inserted at 26-28 weeks' gestation, can be considered for infants with isolated CDH with poor prognosis. This procedure can be minimally invasive, may reverse pulmonary hypoplasia changes, and may improve survival rate in these highly selected cases. In addition, the airways can be restored before birth. In experimental cases studies with animals, fetal tracheal occlusion (TO) induces lung growth and morphologic maturation. Fetoscopic TO with a clip may lead to accelerated lung growth and prevent pulmonary hypoplasia.

There are other spin-off applications which may be achievable utilizing the present invention. Essentially the field of fetal interventional therapy is in an infant stage of development in terms of methods, techniques, devices, drugs, and therapeutic efficacy. For example, the methods described herein could be used for delivery of genetic material in the lung in fetuses with cystic fibrosis, the most common genetic disease in Caucasians.

These and other advantageous features of the present invention will be in part apparent and in part pointed out herein below.

### BRIEF DESCRIPTION OF THE DRAWINGS

For a better understanding of the present invention, reference may be made to the accompanying drawings in which:

FIGS. 1A, 1B, 1C and 1D are an illustration of an overall method and apparatus for tracheal occlusion; and

FIG. 2 is an illustration of an apparatus for tracheal occlusion.

While the invention is susceptible to various modifications and alternative forms, specific embodiments thereof are shown by way of example in the drawings and will herein be described in detail. It should be understood, however, that the drawings and detailed description presented herein are not intended to limit the invention to the particular embodiment disclosed, but on the contrary, the intention is to cover all modifications, equivalents, and alternatives falling within the spirit and scope of the present invention as defined by the appended claims.

### DETAILED DESCRIPTION OF INVENTION

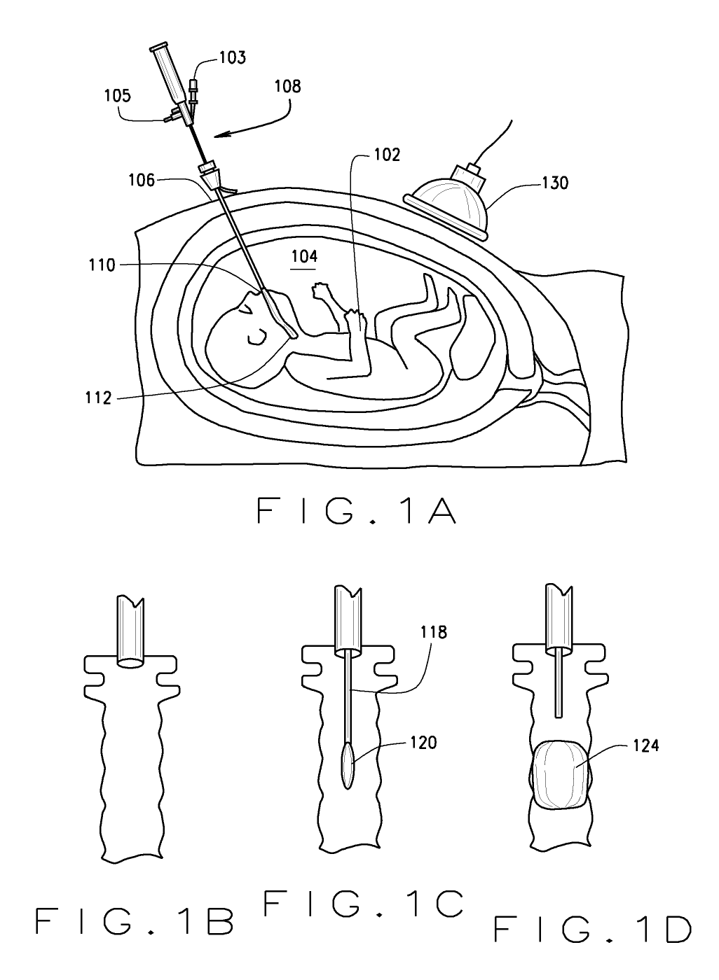

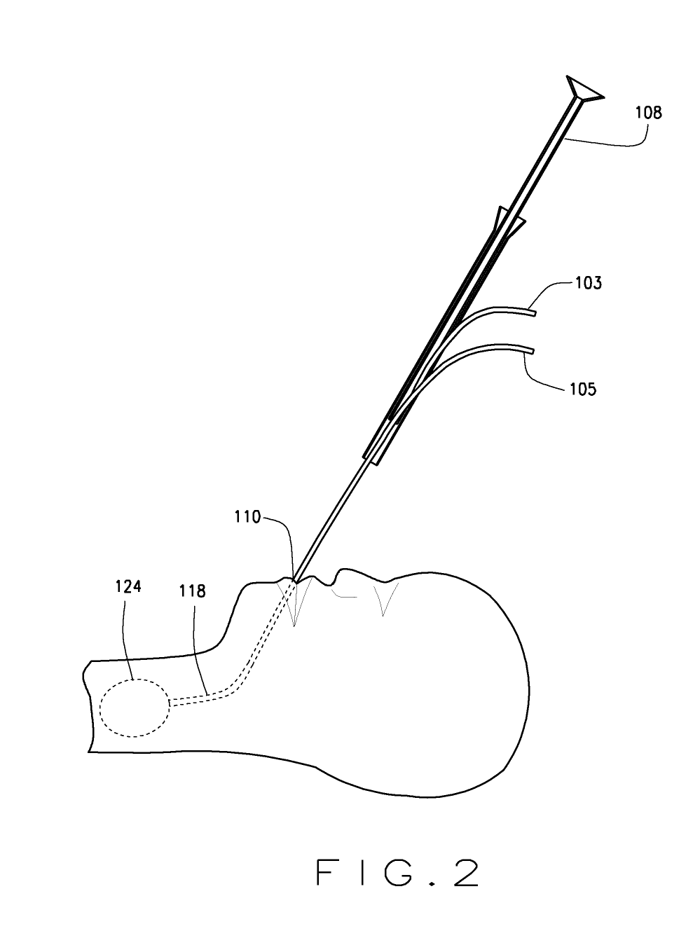

According to the embodiment(s) of the present invention, various views are illustrated in FIG. 1-2 and like reference numerals are being used consistently throughout to refer to like and corresponding parts of the invention for all of the various views and figures of the drawing. Also, please note that the first digit(s) of the reference number for a given item or part of the invention should correspond to the Fig. number in which the item or part is first identified.

One embodiment of the present invention includes (1) the use of a working sheath as a mechanical means of accessing the fetal trachea for drug or device deployment; (2) the use of a temporary occlusion balloon catheter to allow deeper penetration of drug or device delivery into the fetal trachea and lung; (3) the use of polymerizable drugs or compounds injected into the fetal lung below an occlusive balloon so as to achieve a clinical goal, whether that be lung growth, drug delivery or other purposes, which teaches a novel apparatus and method for treating pulmonary hypoplasia or other drug delivery.

The details of the invention and various embodiments can be better understood by referring to the figures of the drawing. Referring to FIGS. 1A, 1B, 1C and 2 a tubular tracheal sheath or fetoscope is utilized to mechanically access the fetal trachea. A dynamic ultra sound scan can be utilize to locate and track the position of the fetus in-utero and utilized to guide the insertion of the instruments. A small incision can be made or a natural canal can be utilized to access the uterus with the instruments. The fetoscope utilized can be a 2.6 mm fetoscope. The fetoscope can be guided through the incision and into the uterus and further inserted into the trachea of the infant. A temporary occlusion balloon catheter with detachable silicone balloon can then be utilized by inserting the catheter through the fetoscope and into the trachea. The balloon can then be inflated and detached from the catheter, which is removed, and the balloon can be left within the trachea. The balloon can be later removed at 34 weeks.

A needle based delivery injection mechanism can then be inserted through the fetoscope and into the trachea where the hydrogel compound can be injected below the balloon. The hydrogel compound can be designed such that it dissolves automatically after a determined period of time. The balloon can also be design for ease of release and removal. Once in place the fluctuations in fluid pressure and amniotic fluid levels can be regulated and moderated.

FIG. 1A illustrates a human fetus 102 positioned within the uterus 104. A small incision 106 has been made to allow for insertion of the fetoscope 108. The fetoscope is inserted through the incision 106 and through the uteral wall. Another method could be to insert the fetoscope through a natural body canal, such as canal 107. The fetoscope 108 can then be guided through the mouth 110 of the human fetus and into the fetal trachea 112 of the fetus. A dynamic ultra sound scan or other imaging system 130 can be utilized to view the interior of the uterus and view the fetus for the purpose of guiding the fetoscope into the trachea 112. The fetoscope can be substantially rigid or can flexible or bendable.

A temporary occlusion balloon catheter 118 with detachable silicone balloon 120 can then be utilized by inserting the catheter 118 through the fetoscope and into the trachea utilizing an instrument access port 103. The balloon 120 can be inflated 124 and detached from the catheter, which is removed, and the balloon can be left within the trachea. A needle based delivery injection mechanism can then be inserted through the fetoscope 108 utilizing a drug delivery access port 105 and into the trachea and pass the balloon, where a hydrogel compound can be injected below the balloon. The hydrogel compound can be designed such that it dissolves automatically after a determined period of time.

The fetoscope can have a rigid or flexible sheath extension or tube. The sheath extension or tube can be designed with the ability to be articulated or maneuvered so that once it is inserted through an incision or canal it can be maneuvered to the desired location. One embodiment of the fetoscope can include a light delivery system to illuminate the organ or object under inspection, in this case illuminate the area around the fetus such that the instrument can be guided to the trachea. The light source is normally outside the body and the light is typically directed via an optical fiber system. A lens system transmitting the image to the viewer from the objective lens to the viewer, typically a relay lens system in the case of rigid endoscopes or a bundle of fiberoptics in the case of a fiberscope. An eyepiece can be included for viewing. However, as indicated above, the instrument can be guided by using an ultrasound system or other imaging device.

An additional internal channel within the sheath or tube can also be provided to allow for insertion and entry of medical instruments or manipulators. The internal channel can be communicably attached to and accessed through and access port as illustrated in one embodiment the access port 103. In this case, a balloon catheter can be inserted through the access port and threaded through the internal channel for deployment. The additional internal channel can also be utilized for delivery of the hydrogel or a separate drug delivery channel can be utilized. A needle injection type system can be used for injecting the hydrogel below the balloon which has been deployed in the trachea of the fetus. The needle injection system can have catheter extending from an injection end of the needle injection system and extendable beyond and below the deployed detached balloon. In a manner similar to that of the balloon catheter, the catheter extending from the injection needle system can be extended and threaded through the internal channel and into the trachea.

The medical instrument for deploying the detachable balloon and the instrument for the hydrogel injection can be combined into one instrument having a balloon catheter and a substantially parallel injection catheter that is extendable beyond and below the detachable balloon. The balloon catheter and the injection catheter can be threaded through the fetoscope concurrently and into the fetal trachea. Another embodiment can utilize two separate internal channels within the fetoscope—one for medical instrument delivery (balloon catheter) and one for on-site drug delivery (injection catheter). Yet another embodiment can include medical device channel for insertion of the balloon catheter and an internal drug delivery catheter with an injection port whereby a standard injection needle containing hydrogel can be utilize by simply inserting the needle into the injection port. These and other related embodiments can be utilized without departing from the scope and spirit of the invention.

The various pulmonary hypoplasia treatment examples shown above illustrate novel method and apparatus for treating pulmonary hypoplasia in infants in-utero. A user of the present invention may choose any of the above methods and apparatus for treating pulmonary hydroplasia, or an equivalent thereof, depending upon the desired application. In this regard, it is recognized that various forms of the subject method and apparatus for treatment as disclosed and discussed herein could be utilized without departing from the spirit and scope of the present invention.

As is evident from the foregoing description, certain aspects of the present invention are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the sprit and scope of the present invention.

Other aspects, objects and advantages of the present invention can be obtained from a study of the drawings, the disclosure and the appended claims.

What is claimed is:

1. A pulmonary treatment method comprising the steps of:
inserting a working sheath fetoscope into a fetal trachea of a fetus in-utero as a mechanical means of accessing the fetal trachea;
inserting a temporary occlusion balloon catheter having a detachable balloon through the working sheath fetoscope and inflating and detaching the detachable balloon in the fetal trachea thereby occluding the fetal trachea;
and
inserting a needle based delivery injection mechanism through the fetoscope and extending into the fetal trachea and injecting a hydrogel compound below the occlusive balloon into the fetal lung.

2. The pulmonary treatment method of claim 1, where the method of treatment is for treating pulmonary hypoplasia so as to achieve a clinical goal of fetal lung growth.

3. The pulmonary treatment method of claim 1, where the method of treatment is for treating pulmonary hypoplasia so as to achieve a clinical goal of drug delivery.

4. The pulmonary treatment method of claim 2, where the hydrogel is a polymerizable hydrogel that will automatically dissolve during the fetal gestation period.

5. The pulmonary treatment method of claim 4, further comprising the step of:
removing the detachable balloon from the fetal trachea.

6. An apparatus for pulmonary treatment comprising:
a working sheath fetoscope sufficiently sized to be inserted through a small incision and into a fetal trachea of a fetus in-utero and having an internal channel within the working sheath fetoscope to allow for insertion and entry of medical instruments as a mechanical means of accessing the fetal trachea;
a temporary occlusion balloon catheter having a detachable balloon and telescopically inserted through the working sheath fetoscope and operable to inflate and detach the detachable balloon in the fetal trachea thereby occluding the fetal trachea;
and
a needle based delivery injection mechanism telescopically inserted through the fetoscope and extendable into the fetal trachea below a detached detachable balloon and said needle based delivery injection mechanism containing an injectable hydrogel compound for delivery below the detached detachable balloon into a fetal lung.

7. The apparatus for pulmonary treatment as recited in claim 6, further comprising:
an internal channel within the working sheath fetoscope to allow for insertion of the temporary occlusion balloon catheter.

8. The apparatus for pulmonary treatment as recited in claim 6, further comprising:
a drug delivery channel within the working sheath fetoscope to allow for insertion of the needle based delivery injection mechanism for injection of the hydrogel.

9. The apparatus for pulmonary treatment as recited in claim 6, further comprising:
an imaging system adapted to provide an image of the fetus in-utero.

10. The apparatus for pulmonary treatment as recited in claim 6, further comprising:
a balloon extraction catheter adapted to extract the detachable balloon and telescopically inserted through the working sheath fetoscope, and where the hydrogel compound has chemical characteristics such that it is a polymerizable hydrogel that will automatically dissolve during the fetal gestation period.
